(19) United States
(12) Patent Application Publication
(10) Pub. No.: US 2026/0216012 A1
(43) Pub. Date: Jul. 30, 2026

(54) OIL-IN-WATER EMULSION

(71) Applicant: L V M H RECHERCHE, Saint Jean De Braye (FR)

(72) Inventors: Masahiro KIKUCHI, Chiyoda-ku, Tokyo (JP); Mai OZAWA, Chiyoda-ku, Tokyo (JP)

(73) Assignee: L V M H RECHERCHE, Saint Jean De Braye (FR)

(21) Appl. No.: 19/143,620

(22) PCT Filed: Dec. 30, 2022
(86) PCT No.: PCT/IB2022/000757

§ 371 Date: Jun. 26, 2025

(51) Int. Cl. A61K 8/06; A61K 8/31; A61K 8/34; A61K 8/365; A61K 8/37; A61K 8/39; A61K 8/73; A61K 8/92; A61K 8/99; A61Q 1/02; A61Q 19/00

(52) U.S. Cl. CPC A61K 8/062; A61K 8/31; A61K 8/345; A61K 8/365; A61K 8/375; A61K 8/39; A61K 8/731; A61K 8/92; A61K 8/99; A61Q 1/02; A61Q 19/00; A61K 2800/28; A61K 2800/48; A61K 2800/805

(57) ABSTRACT

The present invention provides an oil-in-water emulsion comprising (a) at least one acid selected from the group consisting of an alpha hydroxy acid (AHA), a beta hydroxy acid (BHA) and a polyhydroxy alkanoic acid (PHA); (b) batyl alcohol; (c) at least one thickener selected from the group consisting of a cellulosic thickener comprising an alkyl group of 10 to 30 carbon atoms, and, a polysaccharide thickener comprising glucose, glucuronic acid and rhamnose as constituent monosaccharides of the main chain; and (d) an oil. A cosmetic method for caring for and/or making-up keratinic materials, comprising the application onto keratinic materials, in particular onto skin, of said oil-in-water emulsion.

### TECHNICAL FIELD

The present invention relates to an oil-in-water emulsion.

### BACKGROUND ART

A polishing serum is a type of peeling serum comprising a peeling agent such as glycolic acid, used to exfoliate dead cells from a skin surface and impart smoothness to the skin surface. Demand for peeling serum is currently increasing, especially in Asia. The current commercially available products, however, are problematic because the peeling agent leaves a sticky residue on the skin. Adding peeling agents has therefore been difficult, especially from the viewpoint of texture.

### Technical Problem

Oils, on the other hand, generally have an effect of reducing sticky residue on skin, and therefore an emulsified peeling serum can impart skin smoothness without leaving a sticky residue. However, since a peeling agent acts as a strong electrolyte, having an effect of rapidly disintegrating emulsions, it is difficult to produce stable emulsions that contain peeling agents. Moreover, peeling agents normally exhibit their peeling effects in a low pH range of 5.5 or lower, and some emulsifiers or thickeners used to stabilize emulsions do not function well under such low pH conditions.

One example of an emulsified composition is the skin care composition described in WO2015013197A1. This composition is an oil-in-water emulsion with an aqueous phase and an oil phase, the aqueous phase containing greater than about 0.5% of an acid in salt form, and the oil-in-water emulsion also including gum and undergoing essentially no change in chemical state or physical uniformity even when exposed to a temperature of about 50° C. for 14 days.

But there is still a need to provide new oil-in-water emulsions containing an acid with a peeling effect and having improved properties in terms of texture and stability.

### Solution to Problem

The Applicant demonstrated that the use of (b) batyl alcohol, and (c) at least one thickener selected from the group consisting of a cellulosic thickener with an alkyl group of 10 to 30 carbon atoms, and, a polysaccharide thickener comprising glucose, glucuronic acid and rhamnose as constituent monosaccharides of the main chain, in oil-in-water emulsions containing acid and oil, answers the expectations, even at low pH (5.5 or lower, for example).

### SUMMARY OF THE INVENTION

So the present invention relates to an oil-in-water emulsion comprising (a) at least one acid selected from the group consisting of an alpha hydroxy acid (AHA), a beta hydroxy acid (BHA) and a polyhydroxy alkanoic acid (PHA), (b) batyl alcohol, (c) at least one thickener selected from the group consisting of a cellulosic thickener with an alkyl group of 10 to 30 carbon atoms, and, a polysaccharide thickener comprising glucose, glucuronic acid and rhamnose as constituent monosaccharides of the main chain, and (d) an oil.

In a particular embodiment, component (a) in the oil-in-water emulsion contains glycolic acid.

In a particular embodiment, component (a) is present in the oil-in-water emulsion at 0.004 mass % or greater.

In a particular embodiment, the pH of the oil-in-water emulsion is 5.5 or lower.

In a particular embodiment, the oil-in-water emulsion further comprises an emulsifier.

In a particular embodiment, the emulsifier other than component (b) in the oil-in-water emulsion is a non-ionic emulsifier.

In a particular embodiment, a non-ionic emulsifier in the oil-in-water emulsion is a polyglycerol fatty acid ester.

In a particular embodiment, the oil-in-water emulsion further comprises a thickener other than component (c).

In a particular embodiment, component (d) in the oil-in-water emulsion also includes a dicaprylyl ether and/or squalane.

In a particular and preferred embodiment, the oil-in-water emulsion is a polishing serum.

The present invention further provides a cosmetic method for caring for keratinic materials comprising the application onto keratinic materials, in particular onto skin, of the oil-in-water emulsion of the present invention.

### Advantageous Effects of Invention

According to the invention it is possible to provide a stable oil-in-water emulsion exhibiting a peeling effect even at low pH (5.5 or lower, for example).

### DETAILED DESCRIPTION OF THE INVENTION

Preferred embodiments of the invention will now be described in detail. However, the present invention is not limited to the described embodiments.

The oil-in-water emulsion of this embodiment comprises (a) at least one acid selected from the group consisting of an alpha hydroxy acid (AHA), a beta hydroxy acid (BHA) and a polyhydroxy alkanoic acid (PHA), (b) batyl alcohol, (c) at least one thickener selected from the group consisting of a cellulosic thickener with an alkyl group of 10 to 30 carbon atoms, and, a polysaccharide thickener comprising glucose, glucuronic acid and rhamnose as constituent monosaccharides of the main chain, and (d) an oil.

The oil-in-water emulsion of the embodiment has an effect of smoothing the surface of skin (a peeling effect) since it comprises component (a), and also has excellent stability even at low pH (such as 5.5 or lower) since it comprises components (b) and (c), and an excellent usage feel without leaving a sticky residue on skin, since it comprises an oil (component (d)).

<Component (a)>

The oil-in-water emulsion of the invention comprises, as component (a), at least one acid selected from the group consisting of an alpha hydroxy acid (AHA), a beta hydroxy acid (BHA) and a polyhydroxy alkanoic acid (PHA), and mixtures thereof. Component (a) in the oil-in-water emulsion of the embodiment functions as a peeling agent.

Examples of alpha hydroxy acids (AHA) include glycolic acid, lactic acid, mandelic acid, malic acid, tartaric acid, citric acid, and mixtures thereof. Examples of beta hydroxy acids (BHA) include salicylic acid and carnitine. Examples of polyhydroxy alkanoic acids (PHA) include gluconolactone and lactobionic acid.

AHA, BHA and PHA are all weak acids and are known to have chemical peeling (skin exfoliating) action. AHA is water-soluble and removes old keratin on the skin surface, promoting turnover of skin. BHA is water-soluble and gently removes sebum and old skin cells, being effective for pimples and sunburn damage. PHA is less irritating than AHA or BHA with high moisturizing power, and is effective for dry skin or for acne, acne scars, skin roughening and atopy in sensitive skin.

The acid used in component (a) in the oil-in-water emulsion of the invention may be of a single type, or a combination of two or more types. Component (a) is preferably glycolic acid, lactic acid, salicylic acid, or a combination of the same, with glycolic acid being especially preferred.

In a particular embodiment, the total content of component (a) in the oil-in-water emulsion of the invention is 0.004 mass % or greater, 0.01 mass % or greater, 0.05 mass % or greater, 0.1 mass % or greater, or 0.5 mass % or greater and 10 mass % or lower, 9 mass % or lower, 8 mass % or lower, 7 mass % or lower, 6 mass % or lower or 5 mass % or lower, for example, with respect to the total amount of the oil-in-water emulsion. In a preferred embodiment, the total content of component (a) is in the range of 0.1 mass % to 10 mass %, and especially 0.5 mass % to 8 mass % or 1 to 5 mass %, with respect to the total amount of the oil-in-water emulsion.

In a particular embodiment, the total content of component (a) in the oil-in-water emulsion of the invention is a content so that the pH of the oil-in-water emulsion is lower than 7.0, such as 6.5 or lower, 6.0 or lower, 5.5 or lower, 5.0 or lower or 4.5 or lower, more preferably it is a content so that the pH of the oil-in-water emulsion is 5.5 or lower, and even more preferably it is a content so that the pH of the oil-in-water emulsion is 3.5 to 5.5.

<Component (b)>

The oil-in-water emulsion of the invention comprises batyl alcohol as component (b). Component (b) in the oil-in-water emulsion of the invention functions as a co-emulsifier, and also as a humectant.

In a particular embodiment, the content of component (b) in the oil-in-water emulsion of the embodiment is 0.01 mass % or greater, 0.03 mass % or greater, 0.05 mass % or greater, 0.1 mass % or greater, 0.15 mass % or greater, 0.2 mass % or greater or 0.25 mass % or greater, and 3.0 mass % or lower, 2.5 mass % or lower, 2.2 mass % or lower, 2.0 mass % or lower, 1.5 mass % or lower, 1.0 mass % or lower or 0.5 mass % or lower, for example, with respect to the total amount of the oil-in-water emulsion. The total content of component (b) is especially in the range of 0.05 mass % to 3.0 mass %, and particularly 0.1 mass % to 2.0 mass % or 0.2 mass % to 1.0 mass % with respect to the total amount of the oil-in-water emulsion.

<Component (c)>

The oil-in-water emulsion of the invention comprises at least one thickener selected from the group consisting of a cellulosic thickener with an alkyl group of 10 to 30 carbon atoms, and, a polysaccharide thickener comprising glucose, glucuronic acid and rhamnose as constituent monosaccharides of the main chain, as component (c). Component (c) in the oil-in-water emulsion of the invention functions as a thickener, and also works together with component (b) to stabilize the oil-in-water emulsion.

In a particular embodiment, the cellulosic thickener with an alkyl group of 10 to 30 carbon atoms is the hydrophobized modified alkyl cellulose having the structural formula (1)

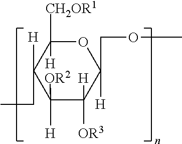

wherein R¹, R² and R³ are the same or different, and are a hydrogen atom, a lower alkyl group a —[CH₂CH₂₋ₖ(CH₃)ₖO]ₘH group, or a —CH₂CH(OH)CH₂OCH₂ⱼ₊₁ group; n is an integer of 100 to 10,000; k is an integer of 0 or 1; m is an integer of 1 to 10; and j is an integer of 6 to 26; and the hydrophobically modified alkyl cellulose necessarily contains a —CH₂CH(OH)CH₂OCH₂ⱼ₊₁ group, wherein the lower alkyl group is a methyl group or an ethyl group, described in WO2011-162421A, for example.

In a particular embodiment, the cellulosic thickener with an alkyl group of 10 to 30 carbon atoms is preferably a cellulosic thickener derived from hydroxypropyl methylcellulose, such as hydroxypropyl methylcellulose stearoxy ether obtained by reacting stearyl glycidyl ether with hydroxypropyl methylcellulose (for example, SANGELOSE 90L by Daido Chemical Corp.). In this case the hydrophobic groups introduced into the hydrophobized modified alkyl cellulose are stearyl groups (—C₁₈H₃₇; carbon number: 18). Reaction may also be carried out using cetyl glycidyl ether and decyl glycidyl ether, instead of stearyl glycidyl ether.

In a particular embodiment, a polysaccharide thickener comprising glucose, glucuronic acid and rhamnose as constituent monosaccharides of the main chain, has a main chain with a structural unit comprising glucose, glucuronic acid, glucose and rhamnose bonded linearly in that order. The polysaccharide thickener of component (c) may also have a side chain as part of the polysaccharide structure, or it may lack a side chain. In a particular embodiment, the side chain has one or more selected from the group consisting of rhamnose, fucose, glucose and mannose as a side chain constituent monosaccharide. The polysaccharide thickener may have a main chain with a structural unit comprising glucose, glucuronic acid, glucose and rhamnose bonded linearly in that order, with rhamnose as a side chain. The polysaccharide thickener may have a main chain with a structural unit comprising glucose, glucuronic acid, glucose and rhamnose bonded linearly in that order, with fucose as a side chain. The polysaccharide thickener may be obtained from a microbe such as a bacterium. According to a specific embodiment, the polysaccharide thickener comprises at least one selected from the group consisting of diutan gum, welan gum, gellan gum, and a combination thereof. Preferably, the polysaccharide thickener is diutan gum. Diutan gum (INCI: Sphingomonas ferment extract) is a polysaccharide obtained by aerobic fermentation of a corn syrup starting material using Alcaligenes microbes.

Component (c) in the oil-in-water emulsion of the embodiment may be of a single type, or a combination of multiple different types. In a particular embodiment, the total content of component (c) with respect to the total amount of the oil-in-water emulsion is 0.01 mass % or greater, 0.02 mass % or greater, 0.03 mass % or greater, 0.05 mass % or greater, 0.06 mass % or greater, 0.07 mass % or greater or 0.1 mass % or greater, and 1.0 mass % or lower, 0.8 mass % or lower, 0.6 mass % or lower, 0.5 mass % or lower, 0.4 mass % or lower, 0.3 mass % or lower or 0.2 mass % or lower, for example. The total content of component (c) is preferably in the range of 0.01 mass % to 1.0 mass % and especially 0.05 mass % to 0.5 mass %, with respect to the total amount of the oil-in-water emulsion.

<Component (d)>

The oil-in-water emulsion of the invention comprises an oil as component (d). In a particular embodiment, the oil of component (d) is in liquid oil or paste oil form, for example, but it is preferably a liquid oil. A liquid oil may be either a non-polar liquid oil or a polar liquid oil.

A liquid oil is a non-volatile oil component that is liquid at ordinary temperature (in the range of 15 to 25° C.) and ordinary pressure (1 atmosphere). Liquid oils include non-volatile hydrocarbon oils, higher alcohols, ester oils, ether oils and vegetable oils.

Examples of non-volatile hydrocarbon oils include squalane, liquid paraffin, vaseline, polybutene, polyisobutene, hydrogenated polyisobutene, hydrogenated polydecene, olefin oligomers, and mixtures thereof.

Higher alcohols are aliphatic monoalcohols of 6 or more carbon atoms, and preferably aliphatic monoalcohols of 12 to 28 carbon atoms. Examples of higher alcohols include octyl alcohol, dodecyl alcohol, oleyl alcohol, octyldodecanol, hexyldecanol, isostearyl alcohol, decyltetradecanol, and mixtures thererof.

Ester oils are carboxylic acid esters that are liquid in environments at ordinary temperature and pressure (for example, 25° C., 1 atmosphere) and do not gasify, and are preferably carboxylic acid alkyl esters wherein the carboxylic acid or alcohol of the ester has 8 to 18 carbon atoms. Examples of esters include propylene glycol fatty acid esters such as propylene glycol dicaprylate and propylene glycol dicaprate; fatty acid alkyl esters such as isoamyl laurate, hexyl laurate, ethylhexyl palmitate, isopropyl myristate, 2-octyldodecyl myristate, cetyl 2-ethylhexanoate, isononyl isononanoate and isotridecyl isononanoate; carbonic acid esters such as dicaprylyl carbonate; tri or greater fatty acid esters of polyglycerol such as polyglyceryl-2 triisostearate, polyglyceryl-2 tetraisostearate and polyglyceryl-10 decaisostearate; glycerol tri fatty acid esters (also known as triglyceride oils) such as glyceryl tri-2-ethylhexanoate, glyceryl tribehenate, glyceryl tri (caprylate/caprate) and triheptanoin; benzoic acid derivatives such as diisostearyl malate and alkyl (C12-15) benzoates; salicylic acid derivatives such as homosalates and ethylhexyl salicylate; alkyl esters such as coconut alkyl (caprylate/caprate) and coconut oil alkyl caprylate; cinnamic acid derivatives such as octyl methoxycinnamate, isopropyl methoxycinnamate, isoamyl methoxycinnamate, cinnoxate, DEA methoxycinnamate, diisopropyl methylcinnamate, glyceryl-ethylhexanoate-dimethoxycinnamate and di-(2-ethylhexyl)-4′-methoxybenzalmalonate; β,β-diphenyl acrylate derivatives such as octocrylene, and mixtures thereof.

Dicaprylyl ether is an example of an ether oil.

Examples of vegetable oils include meadowfoam seed oil, olive fruit oil, jojoba seed oil, camellia oil, coconut oil, macadamia nut oil, rosehip oil, avocado oil, sunflower seed oil, rice bran oil, castor oil, almond oil, and mixtures thereof.

Component (d) in the oil-in-water emulsion of the embodiment may be of a single type, or a combination of multiple different types. Component (d) preferably includes dicaprylyl ether and/or squalane.

In a particular embodiment, the total content of component (d) with respect to the total amount of the oil-in-water emulsion is 1 mass % or greater, 2 mass % or greater, 3 mass % or greater, 4 mass % or greater, 5 mass % or greater, 6 mass % or greater or 7 mass % or greater, and 15 mass % or lower, 14 mass % or lower, 13 mass % or lower, 12 mass % or lower, 11 mass % or lower, 10 mass % or lower, 9 mass % or lower or 8 mass % or lower, for example. The total content of component (d) is preferably in the range of 1 mass % to 15 mass % and especially 2 mass % to 10 mass %, with respect to the total amount of the oil-in-water emulsion.

### <Emulsifier>

According to a specific embodiment, the oil-in-water emulsion may also comprise an emulsifier. Examples of emulsifiers include non-ionic emulsifiers (nonionic surfactants).

Examples of non-ionic emulsifiers include esters of polyglycerol and fatty acids (polyglycerol fatty acid esters) and esters of glycerols and fatty acids (glycerol fatty acid esters), with polyglycerol fatty acid esters being preferred.

According to a specific embodiment, a polyglycerol fatty acid ester is a polyglycerol ester of a fatty acid with 12 to 24, preferably 16 to 22, more preferably 18 to 22 and most preferably 18 or 22 carbon atoms. Examples for constituent fatty acids include palmitic acid, palmitoleic acid, stearic acid, oleic acid, linoleic acid, α-linolenic acid, γ-linolenic acid, isostearic acid, arachidic acid, arachidonic acid and behenic acid. The fatty acid may be saturated or unsaturated, but it is preferably saturated. The number of glycerol units in the polyglycerol fatty acid ester (also referred to as the polymerization degree of the glycerol), may be 2 to 12, preferably 2 to 10 and more preferably 2 to 8, for example, with 2 or 6 being especially preferred.

According to a specific embodiment, the polyglycerol fatty acid ester is selected from the group consisting of polyglyceryl-2 stearate, polyglyceryl-2 isostearate, polyglyceryl-2 oleate, polyglyceryl-6 stearate, polyglyceryl-6 behenate, and mixtures thereof.

According to a specific embodiment, a glycerol fatty acid ester is a glycerol ester of a fatty acid with 12 to 24, preferably 16 to 22, more preferably 16 to 20 and most preferably 18 carbon atoms. Examples of fatty acids include palmitic acid, palmitoleic acid, stearic acid, oleic acid, linoleic acid, α-linolenic acid, γ-linolenic acid, arachidic acid, arachidonic acid and behenic acid. The fatty acid may be saturated or unsaturated, but it is preferably saturated. According to a specific embodiment, the glycerol fatty acid ester is selected from the group consisting of glyceryl stearate, glyceryl oleate, glyceryl myristate, glyceryl coconut fatty acid esters, glyceryl undecylenate, and mixtures thereof.

An emulsifier is preferably a solid at ordinary temperature (for example, 25° C.). According to a particularly preferred embodiment, the emulsifier is preferably selected from the group consisting of polyglycerol esters of fatty acids of 12 to 24 carbon atoms and glycerol esters of fatty acids of 12 to 24 carbon atoms, having 2 to 12 glycerol units, and more preferably it is selected from among polyglycerol esters of fatty acids of 12 to 24 carbon atoms, having 2 to 12 glycerol units, and mixtures thereof. In a particular embodiment, the emulsifier is polyglyceryl-6 stearate or polyglyceryl-6 behenate, for example. An example of a commercial product comprising a combination of polyglyceryl-6 stearate and polyglyceryl-6 behenate is TEGO CARE PBS 6 (Evonik Operations GmbH).

According to a specific embodiment, the emulsifier may be of a single type, or a combination of multiple different types. In a particular embodiment, the total content of the emulsifier with respect to the total amount of the oil-in-water emulsion is 0.1 mass % or greater, 0.2 mass % or greater, 0.5 mass % or greater, 0.7 mass % or greater, 1.0 mass % or greater or 1.2 mass % or greater, and 10 mass % or lower, 8 mass % or lower, 6 mass % or lower, 5 mass % or lower, 4 mass % or lower, 3 mass % or lower, 2.5 mass % or lower, 2.2 mass % or lower or 2 mass % or lower. The total content of emulsifiers with respect to the total amount of the oil-in-water emulsion is especially in the range of 0.5 mass % to 10 mass %, and particularly 0.5 mass % to 5 mass % or preferably 1 mass % to 3 mass %.

### <Thickener>

According to a specific embodiment, the oil-in-water emulsion further includes a thickener other than component (c). Examples of thickeners other than component (c) include cellulosic thickeners other than component (c), polysaccharide thickeners other than component (c), alkyl-modified carboxyvinyl polymers such as Acrylates/C10-30 Alkyl Acrylate Crosspolymer, and acrylic polymers (and (meth)acrylic (co) polymers), and mixtures thereof, preferably polysaccharide thickeners other than component (c), alkyl-modified carboxyvinyl polymers such as Acrylates/C10-30 Alkyl Acrylate Crosspolymer, and acrylic polymers (and (meth)acrylic (co) polymers), and mixtures thereof.

Examples of cellulosic thickeners other than component (c) include ethyl cellulose, hydroxyethyl cellulose, hydroxypropyl cellulose, hydroxypropyl methylcellulose, hydroxypropyl methylcellulose stearoxy ether, cellulose gum, carboxymethyl cellulose, and mixtures thereof.

Polysaccharide thickeners other than component (c) include glucomannan, xanthan gum, dehydroxanthan gum or a mixture of these, for example.

A (meth)acrylic polymer may be a polymer obtained by polymerizing one or more monomers selected from the group consisting of (meth)acrylic acid and (meth)acrylic acid esters. There are no particular restrictions on the (meth)acrylic acid ester, which may be an alkyl (meth)acrylic acid ester, or the same wherein the alkyl group of the alkyl (meth)acrylic acid ester has been substituted with a substituent such as a hydroxyl group, but it is preferably an alkyl (meth)acrylic acid ester. Alkyl (meth)acrylic acid esters include alkyl (meth)acrylic acid esters with alkyl groups of 10 to 30 carbon atoms and alkyl (meth)acrylic acid esters with alkyl groups of 1 to 9 carbon atoms, and are preferably alkyl (meth)acrylic acid esters with alkyl groups of 10 to 30 carbon atoms. In a particular embodiment, a (meth)acrylic polymer is a copolymer of at least one monomer selected from the group consisting of (meth)acrylic acid and alkyl (meth)acrylic acid esters with alkyl groups of 1 to 9 carbon atoms, and at least one monomer selected from the group consisting of alkyl (meth)acrylic acid esters with alkyl groups of 10 to 30 carbon atoms.

In a particular embodiment, a (meth)acrylic polymer also includes a carbomer. Commercially available (meth)acrylic polymers may be used, examples of which include Carbopol™ Ultrez 30 Polymer, Carbopol™ Ultrez 10, Carbopol™ ETD 2020, Carbopol™ Ultrez 20 Polymer and Pemulen TR-1 by Lubrizol, Aristoflex™ AVC by Clariant Japan and Simulgel™ NS MB by Seppic S.A.

According to a specific embodiment, the thickener other than component (c) may be of a single type, or a combination of two or more different types. In a particular embodiment, the total content of the thickener other than component (c) is 0.01 mass % or greater, 0.02 mass % or greater, 0.03 mass % or greater, 0.05 mass % or greater, 0.07 mass % or greater, 0.10 mass % or greater or 0.12 mass % or greater, and 1.0 mass % or lower, 0.8 mass % or lower, 0.5 mass % or lower, 0.3 mass % or lower or 0.2 mass % or lower, for example, with respect to the total amount of the oil-in-water emulsion. The total content of the thickener other than component (c) is preferably in the range of 0.01 mass % to 1.0 mass % with respect to the total amount of the oil-in-water emulsion.

In a particular embodiment, the total content of component (c) and a thickener other than component (c) with respect to the total amount of the oil-in-water emulsion is 0.01 mass % or greater, 0.02 mass % or greater, 0.03 mass % or greater, 0.05 mass % or greater, 0.1 mass % or greater, 0.2 mass % or greater or 0.3 mass % or greater, and 1.5 mass % or lower, 1.0 mass % or lower, 0.8 mass % or lower, 0.6 mass % or lower, 0.5 mass % or lower, 0.4 mass % or lower, 0.3 mass % or lower, 0.2 mass % or lower, 0.15 mass % or lower or 0.1 mass % or lower, for example. The total content of component (c) and the thickener other than component (c) is preferably in the range of 0.01 to 1.5 mass % with respect to the total amount of the oil-in-water emulsion.

### <Humectant>

According to a specific embodiment, the oil-in-water emulsion also comprises a humectant other than component (b). By including a humectant, it is possible to simultaneously provide a high moisturizing effect in addition to the peeling effect. Examples of humectants other than component (b) include polyhydric alcohols of 3 to 5 carbon atoms. Examples of polyhydric alcohols include those with 2 or 3 alcoholic hydroxyl groups. Examples of polyhydric alcohols of 3 to 5 carbon atoms include glycerol, 1,3-butylene glycol, pentylene glycol, propanediol, and mixtures thereof.

According to a specific embodiment, the humectant other than component (b) may be of a single type, or a combination of two or more different types. In a particular embodiment, the total content of the humectant other than component (b) with respect to the total amount of the oil-in-water emulsion is 2 mass % or greater, 3 mass % or greater, 4 mass % or greater, 5 mass % or greater, 6 mass % or greater, 7 mass % or greater, 8 mass % or greater or 9 mass % or greater, and 25 mass % or lower, 20 mass % or lower, 18 mass % or lower, 15 mass % or lower, 13 mass % or lower, 12 mass % or lower, or 11 mass % or lower. The total content of the humectant other than component (b) is most preferably in the range of 2 mass % to 20 mass % and especially 4 mass % to 15 mass %, with respect to the total amount of the oil-in-water emulsion.

### <Other Components>

According to a specific embodiment, the oil-in-water emulsion also contains additives commonly used in cosmetics, such as pH adjustors, perfumes, conservatives, active components, antioxidants, skin whitening agents, pigments, coloring agents, and skin conditioning agents. According to a specific embodiment, the oil-in-water emulsion comprises an active component.

The oil-in-water emulsion of the invention also contains water. In a particular embodiment, the content of water with respect to the total amount of the oil-in-water emulsion is 25 mass % or greater, 30 mass % or greater, 35 mass % or greater, 40 mass % or greater, 45 mass % or greater, 48 mass % or greater or 50 mass % or greater, and 70 mass % or lower, 65 mass % or lower, 60 mass % or lower, 61 mass % or lower, 59 mass % or lower or 57 mass % or lower, for example. In a preferred embodiment, the total water content is in the range of 30 mass % to 70 mass % and especially 35 mass % to 65 mass %, with respect to the total amount of the oil-in-water emulsion.

In a particular embodiment, the pH of the oil-in-water emulsion of the invention is lower than 7.0, and may be 6.5 or lower, 6.0 or lower or 5.5 or lower, and 2.0 or higher, 2.5 or higher, 3.0 or higher or 3.5 or higher, for example. The pH of the oil-in-water emulsion of the invention is most preferably in the range of 3.5 to 5.5.

According to a specific embodiment, the oil-in-water emulsion has a viscosity of 300 to 5,000 mPa·s, 500 to 3,000 mPa·s or 600 to 2,000, for example, as measured at 25° C. The viscosity can be measured based on the shear viscosity, using a rotational viscometer (for example, Rheolab QC by Anton Paar, spindle: ST-22-4V-40; rotational speed: 100 rpm). The oil-in-water emulsion of the invention has relatively low viscosity and a light texture.

According to a specific embodiment, the oil-in-water emulsion is produced, for example, by stirring and mixing component (b), component (c), component (d) and the other thickener, emulsifier, humectant or water, while heating at 80 to 85° C., emulsifying the mixture, and then cooling to room temperature and mixing in component (a) and the other components.

According to another specific embodiment, the oil-in-water emulsion of the embodiment is in the form of a cream or serum, for example. In a particular embodiment, the oil-in-water emulsion of the invention is especially a polishing serum.

The oil-in-water emulsified cosmetic according to a specific embodiment has excellent long-term storage stability (for example, it does not undergo phase separation when stored for 1 month at 50° C.). As a harsh stability test to predict the long-term product stability, evaluation may be carried out by observing the condition of a sample stored for a certain period such as 1 month or 3 months, at a constant temperature of 45° C. or 50° C. For more accurate assessment of the stability, the sample may be centrifuged (a centrifuge tube filled with 10 mL of emulsion is spun at 4200 rpm for 15 minutes in an EPPENDORF centrifuge 5702R, for example) and evaluated.

The present invention will now be explained in detail by examples. However, the invention is not limited to these examples.

### EXAMPLES

### [Preparation of Oil-In-Water Emulsions]

Oil-in-water emulsions of Examples 1 to 9 and Comparative Examples 1 to 12 were prepared with the ingredients and mixing ratios listed in Table 1 to Table 3. The “%” values in Tables 2 and 3 are all wt % values, based on the total weight of each oil-in-water emulsion. The ingredients of Phases D, E, H, and K were dissolved at room temperature. The ingredients of Phase A were dissolved at 80° C. A 0.5 to 1.0 kg portion of the oil-in-water emulsion was prepared using a Rayneri agitator. The ingredients of Phase B were added to a tank and the tank was heated at 80° C. and mixed at 100-500 rpm. After the bulk components in the tank became homogeneous, the ingredients of Phase C were added into the tank and mixed for 20 minutes until the mixture swelled. The ingredients of Phase A were then added into the tank gradually and the mixture in the tank was emulsified with a Rayneri agitator for 5 min at 80° C., and then with a Turrax homogenizer for 1 min at 10,000 rpm at room temperature. The mixture was air-cooled down to 30° C., and the ingredients of the remaining phases were added in order at below 40° C. The oil-in-water emulsions of Examples 1 to 9 and Comparative Examples 1 to 12 were obtained.

TABLE 1

| | | INGREDIENT | |
|---|---|---|---|
| Phase | Function | Product Name/Compound Name | INCI Name/Compound Name |
| A | oil | CETIOL OE MB | DICAPRYLYL ETHER |
| A | oil | SQUALANE VEGETAL | SQUALANE |
| A | oil | TRI CAPRAT/CAPRYLAT GLYCEROL | CAPRYLIC/CAPRIC TRIGLYCERIDE |
| A | oil | POLYGLYCERYL-2 TRIISOSTEARATE MB | POLYGLYCERYL-2 TRIISOSTEARATE |
| A | Non-ionic | TEGO CARE PBS 6 | POLYGLYCERYL-6 STEARATE and |
| | emulsifier | | POLYGLYCERYL-6 BEHENATE |
| A | Co-emulsifier | NIKKOL BATYL ALCOHOL 100 | BATYL ALCOHOL |
| A | Co-emulsifier | TEGIN 90 VEG | GLYCERYL STEARATE |
| A | Co-emulsifier | ALCOOL STEARYLIQUE MB | STEARYL ALCOHOL |
| B | | WATER | WATER |
| B | Humectant | GLYCEROL VEGETAL | GLYCERIN |
| B | Humectant | 1,3 BUTYLENE GLYCOL VEGETAL | BUTYLENE GLYCOL |
| B | Preservative | CHLORPHENESIN | CHLORPHENESIN |
| C | Anionic | DERMOFEEL GSC | GLYCERYL STEARATE CITRATE |
| | emulsifier | | |
| C | Thickener | CARBOPOL ULTREZ 30 | CARBOMER |
| C | Thickener | SANGELOSE 90L | HYDROXYPROPYL METHYLCELLULOSE |
| | | | STEAROXY ETHER |
| C | Thickener | NATROSOL 250M | HYDROXYETHYL CELLULOSE |
| C | Thickener | METOLOSE 90 SH 4000 | HYDROXYPROPYL METHYLCELLULOSE |
| C | Thickener | RHODICARE XC | XANTHAN GUM |
| C | Thickener | MANNAGURUKO HV | GLUCOMANNAN |
| C | Thickener | KELCO-CARE DIUTAN GUM | SPHINGOMONAS FERMENT EXTRACT |
| D | | WATER | WATER |
| D | Neutralizer | SODIUM HYDROXIDE | |
| E | | WATER | WATER |
| E | Neutralizer | SODIUM HYDROXIDE | |
| F | Preservative | PENTYLENE GLYCOL VEG | |
| G | Active | Active ingredients | |
| | ingredient | | |
| H | | WATER | WATER |
| H | Neutralizer | SODIUM HYDROXIDE | |
| I | Active | Active ingredients | |
| | ingredient | | |
| J | Peeling agent | GLYPURE 70 | 70% aqueous solution of GLYCOLIC ACID |
| K | | WATER | WATER |
| K | Neutralizer | SODIUM HYDROXIDE | |
| L | Fragrance | Fragrance | |

TABLE 2

| | | | Comparative | Comparative | | | |
|---|---|---|---|---|---|---|---|
| | | Example 1 | Example 1 | Example 2 | Example 2 | Example 3 | Example 4 |
| Function | INGREDIENT | (%) | (%) | (%) | (%) | (%) | (%) |
| oil | CETIOL OE MB | 4.8000 | 4.8000 | 4.8000 | 4.8000 | 4.8000 | 4.8000 |
| oil | SQUALANE | 2.4000 | 2.4000 | 2.4000 | 2.4000 | 2.4000 | 2.4000 |
| | VEGETAL | | | | | | |
| oil | TRI | | | | | | |
| | CAPRAT/CAPRYLAT | | | | | | |
| | GLYCEROL | | | | | | |
| oil | POLYGLYCERYL-2 | | | | | | |
| | TRIISOSTEARATE | | | | | | |
| | MB | | | | | | |
| Non-ionic | TEGO CARE PBS 6 | 1.5000 | 1.5000 | 1.5000 | 1.5000 | 1.5000 | 1.5000 |
| emulsifier | | | | | | | |
| Co- | NIKKOL BATYL | 0.3000 | 0.3000 | 0.3000 | 0.3000 | 0.3000 | 0.3000 |
| emulsifier | ALCOHOL 100 | | | | | | |
| Co- | TEGIN 90 VEG | | | | | | |
| emulsifier | | | | | | | |
| Co- | ALCOOL | | | | | | |
| emulsifier | STEARYLIQUE MB | | | | | | |
| | WATER | qsp | qsp | qsp | qsp | qsp | qsp |
| Humectant | GLYCEROL | 6.0000 | 6.0000 | 6.0000 | 6.0000 | 6.0000 | 6.0000 |
| | VEGETAL | | | | | | |
| Humectant | 1,3 BUTYLENE | 3.0000 | 3.0000 | 3.0000 | 3.0000 | 3.0000 | 3.0000 |
| | GLYCOL VEGETAL | | | | | | |
| Preservative | CHLORPHENESIN | 0.2700 | 0.2700 | 0.2700 | 0.2700 | 0.2700 | 0.2700 |
| Anionic | DERMOFEEL GSC | 0.3000 | 0.3000 | 0.3000 | 0.3000 | 0.3000 | 0.3000 |
| emulsifier | | | | | | | |
| Thickener | CARBOPOL | 0.3000 | 0.3000 | 0.3000 | 0.3000 | 0.3000 | 0.3000 |
| | ULTREZ 30 | | | | | | |
| Thickener | SANGELOSE 90L | 0.1200 | | | 0.1200 | 0.2100 | |
| Thickener | NATROSOL 250 M | | 0.1200 | | | | |
| Thickener | METOLOSE 90 SH | | | 0.1200 | | | |
| | 4000 | | | | | | |
| Thickener | RHODICARE XC | | | | 0.0900 | | |
| Thickener | MANNAGURUKO | 0.0900 | 0.0900 | 0.0900 | | | |
| | HV | | | | | | |
| Thickener | KELCO-CARE | 0.0300 | 0.0300 | 0.0300 | 0.0300 | 0.0300 | 0.2400 |
| | DIUTAN GUM | | | | | | |
| | WATER | 5.0000 | 5.0000 | 5.0000 | 5.0000 | 5.0000 | 5.0000 |
| Neutralizer | SODIUM | | | | | | |
| | HYDROXIDE | | | | | | |
| | WATER | 3.0000 | 3.0000 | 3.0000 | 3.0000 | 3.0000 | 3.0000 |
| Neutralizer | SODIUM | 0.0600 | 0.0600 | 0.0600 | 0.0600 | 0.0600 | 0.0600 |
| | HYDROXIDE | | | | | | |
| Preservative | PENTYLENE | 3.0000 | 3.0000 | 3.0000 | 3.0000 | 3.0000 | 3.0000 |
| | GLYCOL VEG | | | | | | |
| Active | Active | 4.0000 | 4.0000 | 4.0000 | 4.0000 | 4.0000 | 5.0000 |
| ingredient | ingredients | | | | | | |
| | WATER | 7.0000 | 7.0000 | 7.0000 | 7.0000 | 7.0000 | 7.0000 |
| Neutralizer | SODIUM | | | | | | |
| | HYDROXIDE | | | | | | |
| Active | Active | 4.9740 | 4.9740 | 4.9740 | 4.9740 | 4.9740 | 5.8150 |
| ingredient | ingredients | | | | | | |
| Peeling | GLYPURE 70 | 2.8000 | 2.8000 | 2.8000 | 2.8000 | 2.8000 | 2.8000 |
| agent | | | | | | | |
| | WATER | 5.0000 | 5.0000 | 5.0000 | 5.0000 | 5.0000 | 5.0000 |
| Neutralizer | SODIUM | 0.9000 | 0.9000 | 0.9000 | 0.9000 | 0.9000 | 0.9000 |
| | HYDROXIDE | | | | | | |
| Fragrance | Fragrance | 0.4000 | 0.4000 | 0.4000 | 0.4000 | 0.4000 | 0.4000 |
| Total | | 100.0000 | 100.0000 | 100.0000 | 100.0000 | 100.0000 | 100.0000 |
| Result | pH | 5.05 | | | 5.02 | 5.05 | 4.93 |
| | Viscosity [cP] | 1,302 | | | 1,351 | 1,772 | 1,065 |
| | Centrifugation | conform | Oil exudate | Oil exudate | conform | conform | conform |
| | Condition at | Stable | Oil exudate | Oil exudate | Stable | Stable | Stable |
| | 50 C × 1M | | | | | | |

| | | | Comparative | Comparative | | |
|---|---|---|---|---|---|---|
| | | Example 5 | Example 3 | Example 4 | Example 6 | Example 7 |
| Function | INGREDIENT | (%) | (%) | (%) | (%) | (%) |
| oil | CETIOL OE MB | 4.8000 | 4.8000 | 4.8000 | 4.8000 | 4.5000 |
| oil | SQUALANE | 2.4000 | 2.4000 | 2.4000 | 2.4000 | 1.5000 |
| | VEGETAL | | | | | |
| oil | TRI | | | | | |
| | CAPRAT/CAPRYLAT | | | | | |
| | GLYCEROL | | | | | |
| oil | POLYGLYCERYL-2 | | | | | |
| | TRIISOSTEARATE | | | | | |
| | MB | | | | | |
| Non-ionic | TEGO CARE PBS 6 | 1.5000 | 1.5000 | 1.5000 | 1.5000 | 0.5000 |
| emulsifier | | | | | | |
| Co- | NIKKOL BATYL | 0.3000 | 0.3000 | 0.3000 | 0.3000 | 0.1000 |
| emulsifier | ALCOHOL 100 | | | | | |
| Co- | TEGIN 90 VEG | | | | | |
| emulsifier | | | | | | |
| Co- | ALCOOL | | | | | |
| emulsifier | STEARYLIQUE MB | | | | | |
| | WATER | qsp | qsp | qsp | qsp | qsp |
| Humectant | GLYCEROL | 6.0000 | 6.0000 | 6.0000 | 6.0000 | 6.0000 |
| | VEGETAL | | | | | |
| Humectant | 1,3 BUTYLENE | 3.0000 | 3.0000 | 3.0000 | 3.0000 | 3.0000 |
| | GLYCOL VEGETAL | | | | | |
| Preservative | CHLORPHENESIN | 0.2700 | 0.2700 | 0.2700 | 0.2700 | 0.2700 |
| Anionic | DERMOFEEL GSC | 0.3000 | 0.3000 | 0.3000 | 0.3000 | 0.1000 |
| emulsifier | | | | | | |
| Thickener | CARBOPOL | 0.3000 | 0.3000 | 0.3000 | 0.3000 | 0.2700 |
| | ULTREZ 30 | | | | | |
| Thickener | SANGELOSE 90L | | | | 0.1200 | 0.0900 |
| Thickener | NATROSOL 250 M | | 0.2100 | | | |
| Thickener | METOLOSE 90 SH | | | 0.2100 | | |
| | 4000 | | | | | |
| Thickener | RHODICARE XC | | | | | |
| Thickener | MANNAGURUKO | | | | 0.0900 | 0.0900 |
| | HV | | | | | |
| Thickener | KELCO-CARE | 0.3600 | 0.0300 | 0.0300 | 0.0300 | 0.0900 |
| | DIUTAN GUM | | | | | |
| | WATER | 5.0000 | 5.0000 | 5.0000 | 5.0000 | 5.0000 |
| Neutralizer | SODIUM | | | | 0.0560 | 0.0500 |
| | HYDROXIDE | | | | | |
| | WATER | 3.0000 | 3.0000 | 3.0000 | | |
| Neutralizer | SODIUM | 0.0600 | 0.0600 | 0.0600 | | |
| | HYDROXIDE | | | | | |
| Preservative | PENTYLENE | 3.0000 | 3.0000 | 3.0000 | 3.0000 | 3.0000 |
| | GLYCOL VEG | | | | | |
| Active | Active | 5.0000 | 4.0000 | 4.0000 | 5.0000 | 5.0000 |
| ingredient | ingredients | | | | | |
| | WATER | 7.0000 | 7.0000 | 7.0000 | 5.0000 | 5.0000 |
| Neutralizer | SODIUM | | | | 0.0500 | 0.0500 |
| | HYDROXIDE | | | | | |
| Active | Active | 5.8150 | 4.9740 | 4.9740 | 5.8150 | 5.8150 |
| ingredient | ingredients | | | | | |
| Peeling | GLYPURE 70 | 2.8000 | 2.8000 | 2.8000 | 2.8000 | 2.8000 |
| agent | | | | | | |
| | WATER | 5.0000 | 5.0000 | 5.0000 | 5.0000 | 5.0000 |
| Neutralizer | SODIUM | 0.9000 | 0.9000 | 0.9000 | 0.9000 | 0.9000 |
| | HYDROXIDE | | | | | |
| Fragrance | Fragrance | 0.4000 | 0.4000 | 0.4000 | 0.1500 | 0.1500 |
| Total | | 100.0000 | 100.0000 | 100.0000 | 100.0000 | 100.0000 |
| Result | pH | 4.88 | | | 4.86 | 4.85 |
| | Viscosity [cP] | 1,673 | | | 1,421 | 824 |
| | Centrifugation | conform | Oil exudate | Oil exudate | conform | conform |
| | Condition at | Stable | Oil exudate | Oil exudate | Stable | Stable |
| | 50 C × 1M | | | | | |

TABLE 3

| | | | Comparative | Comparative | Comparative | Comparative | Comparative |
|---|---|---|---|---|---|---|---|
| | | Example 8 | Example 5 | Example 6 | Example 7 | Example 8 | Example 9 |
| Function | INGREDIENT | (%) | (%) | (%) | (%) | (%) | (%) |
| oil | CETIOL OE MB | 4.5000 | 4.5000 | 4.5000 | | 4.5000 | 4.5000 |
| oil | SQUALANE | 1.5000 | 1.5000 | | 1.5000 | 1.5000 | 1.5000 |
| | VEGETAL | | | | | | |
| oil | TRI | | | | 4.5000 | | |
| | CAPRAT/CAPRYLAT | | | | | | |
| | GLYCEROL | | | | | | |
| oil | POLYGLYCERYL-2 | | | 1.5000 | | | |
| | TRIISOSTEARATE | | | | | | |
| | MB | | | | | | |
| Non-ionic | TEGO CARE PBS 6 | 0.5000 | 1.2000 | 1.2000 | 0.5000 | 0.5000 | 0.5000 |
| emulsifier | | | | | | | |
| Co- | NIKKOL BATYL | 0.1000 | 0.3000 | 0.3000 | 0.1000 | 0.1000 | 0.1000 |
| emulsifier | ALCOHOL 100 | | | | | | |
| Co- | TEGIN 90 VEG | | | | | | |
| emulsifier | | | | | | | |
| Co- | ALCOOL | | | | | | |
| emulsifier | STEARYLIQUE MB | | | | | | |
| | WATER | qsp | qsp | qsp | qsp | qsp | qsp |
| Humectant | GLYCEROL | 6.0000 | 6.0000 | 6.0000 | 6.0000 | 6.0000 | 6.0000 |
| | VEGETAL | | | | | | |
| Humectant | 1,3 BUTYLENE | 3.0000 | 3.0000 | 3.0000 | 3.0000 | 3.0000 | 3.0000 |
| | GLYCOL VEGETAL | | | | | | |
| Preservative | CHLORPHENESIN | 0.2700 | 0.2700 | 0.2700 | 0.2700 | 0.2700 | 0.2700 |
| Anionic | DERMOFEEL GSC | 0.1000 | 0.1000 | 0.1000 | 0.1000 | 0.1000 | 0.1000 |
| emulsifier | | | | | | | |
| Thickener | CARBOPOL ULTREZ | 0.2700 | 0.2700 | 0.2700 | 0.2700 | 0.2700 | 0.2700 |
| | 30 | | | | | | |
| Thickener | SANGELOSE 90L | 0.0600 | | | | | |
| Thickener | NATROSOL 250 M | | | | | | |
| Thickener | METOLOSE 90 SH | | | | | | |
| | 4000 | | | | | | |
| Thickener | RHODICARE XC | 0.1200 | 0.1200 | 0.1200 | 0.1200 | 0.1200 | |
| Thickener | MANNAGURUKO HV | | 0.0900 | 0.0900 | 0.0900 | 0.0900 | 0.0900 |
| Thickener | KELCO-CARE | 0.0900 | 0.0600 | 0.0600 | 0.0600 | 0.0600 | 0.1800 |
| | DIUTAN GUM | | | | | | |
| | WATER | 5.0000 | 5.0000 | 5.0000 | 5.0000 | 5.0000 | 5.0000 |
| Neutralizer | SODIUM | 0.0500 | 0.0500 | 0.0500 | 0.0500 | 0.0500 | 0.0500 |
| | HYDROXIDE | | | | | | |
| | WATER | | | | | | |
| Neutralizer | SODIUM | | | | | | |
| | HYDROXIDE | | | | | | |
| Preservative | PENTYLENE | 3.0000 | 3.0000 | 3.0000 | 3.0000 | 3.0000 | 3.0000 |
| | GLYCOL VEG | | | | | | |
| Active | Active | 5.0000 | 5.0000 | 5.0000 | 5.0000 | 5.0000 | 5.0000 |
| ingredient | ingredients | | | | | | |
| | WATER | 5.0000 | 5.0000 | 5.0000 | 5.0000 | 5.0000 | 5.0000 |
| Neutralizer | SODIUM | 0.0500 | 0.0500 | 0.0500 | 0.0500 | 0.0500 | 0.0500 |
| | HYDROXIDE | | | | | | |
| Active | Active | 5.8150 | 5.8150 | 5.8150 | 5.8150 | 5.8150 | 5.8150 |
| ingredient | ingredient | | | | | | |
| Peeling | GLYPURE 70 | 2.8000 | 2.8000 | 2.8000 | 2.8000 | 2.8000 | 2.8000 |
| agent | | | | | | | |
| | WATER | 5.0000 | 5.0000 | 5.0000 | 5.0000 | 5.0000 | 5.0000 |
| Neutralizer | SODIUM | 0.9000 | 0.9000 | 0.9000 | 0.9000 | 0.9000 | 0.9000 |
| | HYDROXIDE | | | | | | |
| Fragrance | Fragrance | 0.1500 | 0.1500 | 0.1500 | 0.1500 | 0.1500 | 0.1500 |
| Total | | 100.0000 | 100.0000 | 100.0000 | 100.0000 | 100.0000 | 100.0000 |
| Result | pH | 4.83 | 4.89 | 4.89 | 4.85 | 4.77 | 4.75 |
| | Viscosity [cP] | 671 | 1,024 | 937 | 637 | 716 | 1,082 |
| | Centrifugation | conform | conform | conform | conform | conform | conform |
| | Condition at | Stable | Not smooth | Not smooth | Not smooth | Not smooth | Not smooth |
| | 50 C × 1M | | Water | Water | Water | | |
| | | | exudate | exudate | exudate | | |

| | | Comparative | | Comparative | Comparative |
|---|---|---|---|---|---|
| | | Example 10 | Example 9 | Example 11 | Example 12 |
| Function | INGREDIENT | (%) | (%) | (%) | (%) |
| oil | CETIOL OE MB | 4.5000 | 6.0000 | 6.0000 | 6.0000 |
| oil | SQUALANE | 1.5000 | 3.0000 | 3.0000 | 3.0000 |
| | VEGETAL | | | | |
| oil | TRI | | | | |
| | CAPRAT/CAPRYLAT | | | | |
| | GLYCEROL | | | | |
| oil | POLYGLYCERYL-2 | | | | |
| | TRIISOSTEARATE | | | | |
| | MB | | | | |
| Non-ionic | TEGO CARE PBS 6 | 0.5000 | 1.5000 | 1.5000 | 1.5000 |
| emulsifier | | | | | |
| Co- | NIKKOL BATYL | 0.1000 | 0.3000 | | |
| emulsifier | ALCOHOL 100 | | | | |
| Co- | TEGIN 90 VEG | | | 0.3000 | |
| emulsifier | | | | | |
| Co- | ALCOOL | | | | 0.3000 |
| emulsifier | STEARYLIQUE MB | | | | |
| | WATER | qsp | qsp | qsp | qsp |
| Humectant | GLYCEROL | 6.0000 | 6.0000 | 6.0000 | 6.0000 |
| | VEGETAL | | | | |
| Humectant | 1,3 BUTYLENE | 3.0000 | 3.0000 | 3.0000 | 3.0000 |
| | GLYCOL VEGETAL | | | | |
| Preservative | CHLORPHENESIN | 0.2700 | 0.2700 | 0.2700 | 0.2700 |
| Anionic | DERMOFEEL GSC | 0.1000 | 0.3000 | 0.3000 | 0.3000 |
| emulsifier | | | | | |
| Thickener | CARBOPOL ULTREZ | 0.2700 | 0.2700 | 0.2700 | 0.2700 |
| | 30 | | | | |
| Thickener | SANGELOSE 90L | | 0.0600 | 0.0600 | 0.0600 |
| Thickener | NATROSOL 250 M | | | | |
| Thickener | METOLOSE 90 SH | | | | |
| | 4000 | | | | |
| Thickener | RHODICARE XC | 0.1800 | 0.1200 | 0.1200 | 0.1200 |
| Thickener | MANNAGURUKO HV | 0.0900 | 0.0900 | 0.0900 | 0.0900 |
| Thickener | KELCO-CARE | | | | |
| | DIUTAN GUM | | | | |
| | WATER | 5.0000 | 5.0000 | 5.0000 | 5.0000 |
| Neutralizer | SODIUM | 0.0500 | 0.0500 | 0.0500 | 0.0500 |
| | HYDROXIDE | | | | |
| | WATER | | | | |
| Neutralizer | SODIUM | | | | |
| | HYDROXIDE | | | | |
| Preservative | PENTYLENE | 3.0000 | 3.0000 | 3.0000 | 3.0000 |
| | GLYCOL VEG | | | | |
| Active | Active | 5.0000 | 5.0000 | 5.0000 | 5.0000 |
| ingredient | ingredients | | | | |
| | WATER | 5.0000 | 5.0000 | 5.0000 | 5.0000 |
| Neutralizer | SODIUM | 0.0500 | 0.0500 | 0.0500 | 0.0500 |
| | HYDROXIDE | | | | |
| Active | Active | 5.8150 | 5.8150 | 5.8150 | 5.8150 |
| ingredient | ingredient | | | | |
| Peeling | GLYPURE 70 | 2.8000 | 2.8000 | 2.8000 | 2.8000 |
| agent | | | | | |
| | WATER | 5.0000 | 5.0000 | 5.0000 | 5.0000 |
| Neutralizer | SODIUM | 0.9000 | 0.9000 | 0.9000 | 0.9000 |
| | HYDROXIDE | | | | |
| Fragrance | Fragrance | 0.1500 | 0.1500 | 0.1500 | 0.1500 |
| Total | | 100.0000 | 100.0000 | 100.0000 | 100.0000 |
| Result | pH | 4.81 | 4.79 | 4.98 | 4.86 |
| | Viscosity [cP] | 467 | 1,571 | 1,613 | 1,962 |
| | Centrifugation | conform | conform | Oil | Water |
| | | | | exudate | exudate |
| | Condition at | Not smooth | Stable | Not smooth | Jelly-like |
| | 50 C × 1M | Water | | Water | Water |
| | | exudate | | exudate | exudate |

The stability of each obtained emulsion was evaluated by the following method. The viscosity was measured as the shear viscosity determined using a viscometer (RheolabQC (Anton Paar), spindle: ST-22-4V-40; rotational speed: 100 rpm) at 25° C.

The stability was evaluated by examining the state of the emulsion after centrifugal separation (4,200 rpm, 15 minutes) and after storage for 1 month at 50° C. The results of the stable test demonstrated that SANGELOSE 90L (hydroxypropyl methylcellulose stearoxy ether) and NIKKOL Batyl Alcohol 100 (batyl alcohol) stabilized the emulsions (Examples 1 to 3, and 6 to 9). When other cellulosic thickeners: hydroxyethyl cellulose (Comparative Examples 1 and 3) or hydroxypropyl methyl cellulose (Comparative Examples 2 and 4) were used instead of hydroxypropyl methylcellulose stearoxy ether, or when no cellulosic thickener was used (Comparative Examples 5 to 10), phase separation was observed and the emulsion could not be stabilized. The emulsion also could not be stabilized when no batyl alcohol was included (Comparative Examples 11 and 12). Furthermore, when diutan gum was used instead of SANGELOSE 90L, the emulsion was stabilized in the same manner as SANGELOSE 90L (Examples 4 and 5). So these results demonstrated that the use of (b) batyl alcohol, and (c) at least one thickener selected from the group consisting of a cellulosic thickener with an alkyl group of 10 to 30 carbon atoms, and, a polysaccharide thickener comprising glucose, glucuronic acid and rhamnose as constituent monosaccharides of the main chain, are able to stabilize an oil-in-water emulsion containing an acid, even at low pH (5.5 and lower).

1. An oil-in-water emulsion comprising:
(a) at least one acid selected from the group consisting of an alpha hydroxy acid (AHA), a beta hydroxy acid (BHA) and a polyhydroxy alkanoic acid (PHA),
(b) batyl alcohol,
(c) at least one thickener selected from the group consisting of a cellulosic thickener with an alkyl group of 10 to 30 carbon atoms, and, a polysaccharide thickener comprising glucose, glucuronic acid and rhamnose as constituent monosaccharides of the main chain, and
(d) an oil.

2. The oil-in-water emulsion according to claim 1, wherein component (a) comprises glycolic acid.

3. The oil-in-water emulsion according to claim 1, which comprises component (a) at 0.004 mass % or greater, with respect to the total amount of the oil-in-water emulsion.

4. The oil-in-water emulsion according to claim 1, wherein the pH is 5.5 or lower.

5. The oil-in-water emulsion according to claim 1, which further comprises an emulsifier.

6. The oil-in-water emulsion according to claim 5, wherein the emulsifier is a non-ionic emulsifier.

7. The oil-in-water emulsion according to claim 6, wherein the non-ionic emulsifier is a polyglycerol fatty acid ester.

8. The oil-in-water emulsion according to claim 1, which further comprises a thickener other than component (c).

9. The oil-in-water emulsion according to claim 1, wherein component (d) comprises dicaprylyl ether and/or squalane.

10. The oil-in-water emulsion according to claim 1, which is a polishing serum.

11. A cosmetic method for caring for and/or making-up keratinic materials, comprising the application onto keratinic materials of the oil-in-water emulsion according to claim 1.

12. The oil-in-water emulsion according to claim 1, which comprises component (a) in the range of 0.1 mass % to 10 mass %, with respect to the total amount of the oil-in-water emulsion.

13. The oil-in-water emulsion according to claim 1, which comprises component (a) in the range of 0.5 mass % to 8 mass % with respect to the total amount of the oil-in-water emulsion.

14. The oil-in-water emulsion according to claim 1, which comprises component (a) in the range of 1 to 5 mass %, with respect to the total amount of the oil-in-water emulsion.

15. A cosmetic method according to claim 11, wherein said keratinic materials is skin.
